(19) United States
(12) Patent Application Publication
(10) Pub. No.: US 2010/0056773 A1
(43) Pub. Date: Mar. 4, 2010

(54) DECOLORIZATION OF PROCESS STREAMS BY CHEMICAL OXIDATION IN THE MANUFACTURE OF TRICHLOROGALACTOSUCROSE

(75) Inventors: Batchu Chandrasekhar, Chennai (IN); Andanagouda Sharanappagouda Patil, Chennai (IN); Rakesh Ratnam, Chennai (IN); Sundeep Aurora, Chennai (IN)

Correspondence Address: THE NATH LAW GROUP, 112 South West Street, Alexandria, VA, 22314, US

(73) Assignee: V.B. Medicare PVT. Ltd., Bangalore (IN)

(21) Appl. No.: 12/448,791

(22) PCT Filed: Jan. 2, 2008
(86) PCT No.: PCT/IN2008/000006

§ 371 Date: Aug. 6, 2009

(30) Foreign Application Priority Data
Jan. 8, 2007 (IN) 42/MUM/2007

(51) Int. Cl. C07H 1/00

(52) U.S. Cl. 536/124

(57) ABSTRACT

A process is described in which decolorization of solutions or reaction mixtures containing trichlorogalactosucrose or 6-acetyl trichlorogalactosucrose is achieved described by bubbling ozone. The method can be used at various stages in the process of production and with or without a combination with other adsorbents for colour removal.

### TECHNICAL FIELD

The present invention relates to decolorization treatment to a process stream during production of chlorinated sugars including 1′-6′-Dichloro-1′-6′-DIDEOXY-β-Fructofuranasyl-4-chloro-4-deoxy-galactopyranoside i.e. trichlorogalactosucrose (TGS) and its precursor (TGS-6-ester).

### BACKGROUND OF THE INVENTION

Majority of strategies used in prior art methods of production of 4,1′,6′ trichlorogalactosucrose, the high intensity sweetener, abbreviated for the purpose of this specification as “TGS”, also expressed as 1′-6′-Dichloro-1′-6′-DIDEOXY-β-Fructofuranasyl-4-chloro-4-deoxy-galactopyranoside, predominantly involve chlorination of 6-O-acyl sucrose by use of Vilsmeier-Haack reagent, to form 6 acyl 4,1′,6′trichlorogalactosucrose, using various chlorinating agents such as phosphorus oxychloride, oxalyl chloride, phosphorus pentachloride etc, and a tertiary amide such as dimethyl formamide (DMF). After the said chlorination reaction, the reaction mass is neutralized to pH 7.0-7.5 using appropriate alkali hydroxides of calcium, sodium, etc. to deacylate/deacetylate the 6 acetyl 4,1′,6′trichlorogalactosucrose to form 4,1′,6′ trichlorogalactosucrose (TGS).

The chlorinated mass after chlorination whether before or after deacylation is very dark in colour. This is due to a large number of decomposition products produced during the chlorination process. These compounds mostly are mixtures of caramel, furfurals etc. These compounds are highly undesirable and impart strong colour on the product that is very difficult to remove selectively by a particular adsorptive process such as carbon treatment etc.

Individual components of the mixture of colour imparting compounds present in the chlorinated mass have a wide variety of properties, and some compounds have properties close to that of TGS. Therefore, these colour compounds move along with the purified TGS till the crystallization stage and impart strong colour on the product crystallized. This results in reduction in product purity and the final product specifications are not achieved easily.

During the purification of TGS by affinity chromatography, these colour compounds which have similar properties to that of TGS also bind along with TGS on to the resin and elute out along with the product. This results in reduction of the adsorptive capacity of the resin and is therefore undesirable.

The colouring compounds, which interfere during crystallization, must be removed by various treatment methods including but not limited to charcoal treatment and the like. In some cases, charcoal treatment is carried out repeatedly to obtain a colour free product.

### SUMMARY OF THE INVENTION

Invention is embodied in a process of removing the colour from the chlorinated mass or any other process stream in the purification cycle of TGS by subjecting the said stream to a chemical oxidation process.

In particular embodiment, The said chemical oxidation process comprises using ozone gas to remove the colour from the process stream without or in addition to subjecting the same to any kind of adsorptive methods. The ozone gas is directly bubbled into the process stream and the decolourisation starts immediately. When the desired colour of the stream is achieved, it is then taken for the next purification stage.

### DETAILED DESCRIPTION OF THE INVENTION

Passing a colored process flow obtained during the production of TGS through a bed of charcoal is the presently prevalent method of removal of colour. This method, however, results in losses of the product in the charcoal bed and presents a problem of disposal of the used charcoal powder.

It was found that bubbling ozone through coloured process flow of TGS provided a very convenient way of removal of colour that was effective as well as resulted in no by-products to be treated or disposed off. Further, there was no loss of TGS during ozonization.

The colour reduction is measured in terms of Colour index Units abbreviated as CU measured on Platinum Cobalt Scale. The typical colour reduction is 6200 units per 1000 L in a time period of 4-5 hrs with an ozone gas bubbling rate at 100-120 g/hr. The gas bubbled as ozone in the preferred process is a gas that effectively contains 7% to 14% of ozone dissolved in oxygen. This decolourisation is accompanied by reduction in pH of the solution and requires pH adjustment during ozonization process.

The decolourisation step can be applied at one or more of a stage of TGS purification process including but not limited to the following:
1. Neutralized mass after chlorination containing 6-acetyl TGS with/without DMF.
2. Deacylated mass after chlorination containing TGS with/without DMF.
3. 6-acetyl TGS/TGS in an organic water immiscible solvent extract including one or more of ethyl acetate, methyl ethyl ketone, butyl acetate, methylene chloride, and the like.
4. 6-acetyl TGS/TGS in water.
5. 6-acetyl TGS/TGS in water+a water miscible solvent including one or more of methanol, acetone and the like.
6. Any partially purified process stream, obtained in a process of preparation of TGS, containing hydrophilic or hydrophobic impurities.
7. A purified product stream prior to crystallization.
8. A crystallized product subjected to colour removal by re-dissolving in aqueous or organic solvent in which TGS/6-acetyl TGS is soluble.
9. Can be used in addition to any adsorptive colour removal not limited to charcoal treatment, resin treatment, etc.
10. Can be used for more than one time in the process.

The acetyl radical in 6-acetyl TGS can be replaced by any other acyl radical, such as benzoyl.

The examples described below serve as an illustration on how to practice the invention claimed in this specification and do not limit the scope of actual techniques used or scope of or range of reaction conditions or process conditions claimed. Several other adaptations of the embodiments will be easily anticipated by those skilled in this art and they are also included within the scope of this work. Several other adaptations of the embodiments will be easily anticipated by those skilled in this art and they are also included within the scope of this priority of subject matter covered by this specification. Throughout this specification, singular also encompasses, wherever applicable in the context, its plural as well as one or more members of the same kind. Thus, “a chlorinated sugar” in the context of claim on a process of production comprises one as well as another as well as more than one chlorinated sugars to which the claim is relevant. Equivalent alternatives of a reactant or a reaction condition are also included within the scope of claims of this specification. Thus, mention of a 6-acetyl sucrose also encompasses in it one or more of a 6-acyl sucrose including 6-bezoyl sucrose, 6-acetyl sucrose, sucrose-6-phthalate, sucrose-6-propionate, sucrose-6-glutarate and the like. In general, any modification or an equivalent obvious to a person skilled in the art is included within the scope of this specification and its claims.

### EXAMPLE 1

### Ozonization of 6-Acetyl TGS Prior to Deacylation and Crystallization of TGS

In one experiment, 70 kg of 6-acetyl sucrose was chlorinated using the Vilsmeier-Haack Reagent generated from thionyl chloride.

386 kg of DMF was taken in a reactor and 16 kg of carbon was added to it. The mass was stirred and 300 kg of thionyl chloride was added dropwise keeping the temperature below 40° C. The mass was stirred for 60 minutes and then cooled to 0-5° C.

65 kg of 6-acetyl sucrose in DMF was added to the Vilsmeier reagent formed keeping the temperature below 5° C. After the addition, the reaction mass temperature was raised to room temperature maintained for 60 minutes.

Then the mass was heated to 85° C. maintained for 60 minutes, heated again to 100° C. maintained for 6 hrs and further heated to 115° C. and maintained for 1.5 hrs.

The chlorinated mass containing 42% 6-acetyl TGS was then quenched with 1:1 calcium hydroxide slurry up to pH 7.0 and the mass was filtered through the filter press. The clear filtrate was then subjected to affinity chromatography using ADS 600 (Thermax India) resin. The filtered mass was passed through the resin bed filled in a column at 300 LPH and the 6-acetyl TGS and other chlorinated derivatives were selectively bound to the resin and the DMF water layer consisting of the inorganic salts in soluble form passed out as a flow-through fraction. The resin was then washed with 2 bed volumes of water at pH 7.0 and then 6-acetyl TGS fractions were eluted out and collected separately carefully using 35% methanol in water. Methanol from the pure 6-acetyl TGS fractions was removed by Falling Film evaporator.

The eluent fraction was then subjected to falling film evaporation to remove the methanol completely. The concentrate was then subjected to ozonization by bubbling ozone gas through the solution. The volume of the solution was 1400 L containing 26.6 kg of 6-acetyl TGS. The initial colour index of the feed was 12500 CU on the Platinum Cobalt scale and the pH of the solution was 5.38. In the preferred process, the ozone gas used for bubbling into the feed was generated as a gas containing high concentration of ozone by using plant of Megazone series Model M212 (M/s. Aurozon P.O. Box 43, Pondicherry, India) Any other ozone plant giving a gas containing ozone at a concentration sufficient to cause decolorization can be used for the purpose of this invention. The bubbling rate was fixed to 150 g/hr of ozone gas with a concentration rating of 7% nominal and maximal up to 14% w/w in oxygen, although other rates of bubbling may also be used.

The pH of the solution was adjusted between 5-6 using 10% sodium hydroxide solution during the ozonization. The ozonization was completed after 14 hrs and the colour index of the solution after ozonization was found to be 65 CU on the Platinum Cobalt scale. The 6acetyl TGS before and after ozonization remained the same without any loss.

This solution was then subjected to deacylation by addition of 35% sodium hydroxide solution to raise the pH up to 9.5 and monitored by TLC under stirring. After deacylation, the solution was subjected to extraction using ethyl acetate and concentrated.

The TGS was then crystallized in a mixture of methanol and ethyl acetate and dried. The purity of the TGS isolated was found to be 99.6% with an overall yield of 34.5%.

### EXAMPLE 2

Ozonization of Deacylated Mass After Chlorination Containing TGS with DMF & Further During Purification

DMF, 595 kg, was taken in a reactor and 316 kg of Phosphorus Pentachloride was added slowly keeping the temperature below 40° C. The vilsmeier reagent was allowed to form and the mass was stirred for 60 minutes and then cooled to 0-5° C.

100 kg of 6-acetyl sucrose in DMF was added to the Vilsmeier reagent formed keeping the temperature below 5° C. After the addition, the reaction mass temperature was raised to room temperature maintained for 60 minutes.

Then the mass was heated to 85° C. maintained for 60 minutes, heated again to 100° C. maintained for 6 hrs and further heated to 115° C. and maintained for 1.5 hrs.

The chlorinated mass containing 6-acetyl TGS was then quenched with 1:1 calcium hydroxide slurry up to pH 9.5 and the mass was held under stirring at 22° C. for 3 hours for completion of deacylation. The deacylation was confirmed by TLC analysis and pH was then adjusted to 7.0 using dilute HCl. The mass containing TGS was filtered through the filter press. The DMF content of the filtrate was found to be 22% and TGS was 43.5 kg.

22 kg equivalent of the above solution was then subjected to ozonization by bubbling ozone gas through the solution. The initial colour index of the feed was very high on the Platinum Cobalt scale and the pH of the solution was adjusted to 5.5. In the preferred process, the ozone gas used for bubbling into the feed was generated as a gas containing high concentration of ozone by using plant of Megazone series Model M212 (M/s. Aurozon P.O. Box 43, Pondicherry, India) The bubbling rate was fixed to 150 g/hr of ozone gas with a concentration rating of 7% nominal and maximal up to 14% w/w in oxygen, although other rates of bubbling may also be used.

The pH of the solution was adjusted between 5-6 using 10% sodium hydroxide solution during the ozonization. The ozonization was completed after 25 hrs and the colour index of the solution after ozonization was found to be 2500 CU on the Platinum Cobalt scale.

The solution was then taken for purification through the affinity chromatographic process ADS 600 (Thermax India) resin. The filtered mass was passed through the resin bed filled in a column at 300 LPH and the TGS and other chlorinated derivatives were selectively bound to the resin and the DMF water layer consisting of the inorganic salts in soluble form passed out as a flow-through fraction. The resin was then washed with 2 bed volumes of water at pH 7.0 and then TGS fractions were eluted out and collected separately carefully using 30% methanol in water. Methanol from the pure TGS fractions was removed by Falling Film evaporator.

The eluent fraction was then subjected to falling film evaporation to remove the methanol completely. The concentrate was then subjected to ozonization by bubbling ozone gas through the solution. The volume of the solution was 1700 L containing 20.2 kg of 6-acetyl TGS. The initial colour index of the feed was 8500 CU on the Platinum Cobalt scale and the pH of the solution was 5.68. In the preferred process, the ozone gas used for bubbling into the feed was generated as a gas containing high concentration of ozone by using plant of Megazone series Model M212 (M/s. Aurozon P.O. Box 43, Pondicherry, India) The bubbling rate was fixed to 150 g/hr of ozone gas with a concentration rating of 7% nominal and maximal up to 14% w/w in oxygen, although other rates of bubbling may also be used.

The pH of the solution was adjusted between 5-6 using 10% sodium hydroxide solution during the ozonization. The ozonization was completed after 12 hrs and the colour index of the solution after ozonization was found to be 45 CU on the Platinum Cobalt scale. The TGS before and after ozonization remained the same without any loss.

This solution was extracted using ethyl acetate and concentrated. The TGS was then crystallized in a mixture of methanol and ethyl acetate and dried. The purity of the TGS isolated was found to be 98.69% with an overall yield of 33.5%.

### EXAMPLE 3

### Ozonization of Ethyl Acetate Extract of Neutralized Mass After Deacylation Containing TGS

21 kg TGS equivalent solution after deacylation from Example 2 was taken for ethyl acetate extraction. 1:3.5 times v/v of deacetylated mass to ethyl acetate was added and stirred well and the layers were allowed to separate. The separated layers were pooled together. The ethyl acetate extract was optionally concentrated under vacuum at 45° C. up to 50% of its initial volume and was subjected to color removal by ozonization.

The initial colour index of the feed was 1000 CU on the Platinum Cobalt scale. In the preferred process, the ozone gas used for bubbling into the feed was generated as a gas containing high concentration of ozone by using plant of Megazone series Model M212 (M/s. Aurozon P.O. Box 43, Pondicherry, India) The bubbling rate was fixed to 150 g/hr of ozone gas with a concentration rating of 7% nominal and maximal up to 14% w/w in oxygen, although other rates of bubbling may also be used.

Sodium bicarbonate crystals were directly added to the solution and stirred to maintain the pH of the solution between 5-6 during ozonization. The ozonization was completed after 20 hrs and the colour index of the solution after ozonization was found to be 150 CU on the Platinum Cobalt scale.

The ethyl acetate extract after ozonization was then washed with 1:0.1 v/v of saturated sodium chloride solution 10 times to remove the DMF from the extract. After removal of DMF, the ethyl acetate extract was further concentrated to maximum and then the syrup obtained was loaded on to silanized silica gel packed in SS column. Sodium acetate buffer at pH 10.5 was used as mobile phase and the pure fractions of TGS was collected. The TGS fractions were concentrated by Reverse Osmosis process and the concentrate obtained was extracted into ethyl acetate. The TGS was then subjected to further color removal by charcoal treatment where in the initial color index was found to be 150 CU on the Platinum Cobalt scale. 2% Charcoal w/v was taken and added to the ethyl acetate extract was stirred and heated to 45° C. and maintained for 30 minutes. The solution was then cooled and filtered and the color index was found to be 32 CU on Platinum Cobalt scale. The crystallization was carried out in a mixture of methanol and ethyl acetate and dried. The purity of the TGS isolated was found to be 99.23%. The overall yield of TGS obtained was about 35%.

1. A process of production of a chlorinated sugar comprising need to remove at least one colored decomposition product of sugar or at least one of its coloured decomposition derivative, wherein the said color is removed by a chemical oxidation process that does not adversely affect the chlorinated sugar.

2. A process of claim 1 where the said chlorinated sugar is trichlorogalactosucrose (TGS).

3. A process of claim 2 wherein the said chemical oxidation process comprises using ozone.

4. A process of claim 3 wherein the said process comprises bubbling ozone through a process flow, preferably maintaining pH between about 5 to 6.

5. A process of claim 4 wherein the said process of production of TGS comprises steps of:
a. chlorination of 6-acyl sucrose by reacting with an acid chloride, with or without dimethylformamide (DMF),
b. deacetylated mass after chlorination containing TGS with/without DMF.

6. A process of claim 4 wherein the process flow or a composition subjected to colour removal is at least one of the following:
a. 6-acyl TGS/TGS in an organic water immiscible solvent,
b. 6-acyl TGS/TGS in water,
c. 6-acyl TGS/TGS in water with a water miscible solvent,
d. a partially purified process stream, obtained in a process of preparation of TGS, containing hydrophilic or hydrophobic impurities,
e. a purified product stream prior to crystallization,
f. a crystallized product subjected to colour removal by re-dissolving in aqueous or organic solvent in which TGS/6-acetyl TGS is soluble.

7. A process of claim 5 when the said process is used, in any sequence:
a. in addition to at least one step of adsorptive colour removal, or/and
b. more than one time in the process.

8. A process of claim 7 when the said absorptive colour removal is by a charcoal treatment, or a resin treatment.

9. A process of claim 8 where 6-acyl TGS comprises 6-acetyl TGS or 6-butyl TGS.

10. A process of production of Trichlorogalactosucrose (TGS) comprising following steps:
a. chlorinating 6-acetyl TGS by vilsmeier reagent accompanied by heating,
b. quenching the chlorination reaction mixture and adjusting the pH to about 7,
c. selectively binding 6-acetyl TGS and other acetyl chlorinated sugars in the reaction mixture on an affinity chromatography resin,
d. eluting out the adsorbed 6-acetyl TGS from the resin,
e. subjecting the eluted out fraction, preferably after concentration, to bubbling of ozone maintaining the pH between 5 to 6, for a period of time enough to reduce colour to acceptably low level, preferably reading around 70 CU units on platinum cobalt scale,
f. deacylation,
g. extraction of TGS in ethyl acetate, and
h. isolation of solid TGS.

11. A process of production of Trichlorogalactosucrose (TGS) comprising following steps:
a. chlorinating 6-acetyl TGS by vilsmeier reagent accompanied by heating,
b. quenching the chlorination reaction mixture and adjusting the pH to about 7,
c. deacetylation,
d. subjecting the deacetylated reaction mixture for ozone bubbling accompanied by maintaining pH between about 5 to 6,
e. selectively binding TGS and other chlorinated sugars in the reaction mixture on an affinity chromatography resin,
f. concentrating and removing volatile solvent by passing through falling film evaporation process,
g. subjecting the concentrated TGS solution in water to ozone bubbling accompanied by maintaining pH between about 5 to 6 by adding NaOH,
h. extracting TGS in ethyl acetate,
i. isolating solid TGS.

12. A process of production of Trichlorogalactosucrose (TGS) comprising following steps:
a. chlorinating 6-acetyl TGS by vilsmeier reagent accompanied by heating,
b. quenching the chlorination reaction mixture and adjusting the pH to about 7,
c. deacetylation,
d. extracting in ethyl acetate,
e. optionally concentrating ethyl acetate extract to about 50% volume under educed pressure,
f. bubbling ozone gas through ethyl acetate extract maintaining pH to about 5 to 6,
g. isolating solid TGS.
